(19) United States
(12) Patent Application Publication
(10) Pub. No.: US 2019/0220715 A1
(43) Pub. Date: Jul. 18, 2019

(54) OBJECT TRACKING AND LOCATING

(71) Applicant: Precision Dynamics Corporation, Valencia, CA (US)

(72) Inventors: Jun Seok Park, La Crescenta, CA (US); Sohail Anwar, New Berlin, WI (US); Bill Meserve, Moorpark, CA (US); Timothy Wei Tie Qian, West Bend, WI (US); John Park, Chatsworth, CA (US); Marisa Fienup, Ventura, CA (US)

(73) Assignee: Precision Dynamics Corporation, Valencia, CA (US)

(21) Appl. No.: 15/874,868

(22) Filed: Jan. 18, 2018

(51) Int. Cl. G06K 17/00; G06K 7/10

(52) U.S. Cl. CPC G06K 17/0022; G06K 7/10297; G16H 40/20; G06K 7/10762; G06K 7/10425

(57) ABSTRACT

The present disclosure describes various systems and methods for object tracking and locating. An example method includes: receiving, from a first mobile device, a first input identifying a first location inside an establishment; receiving, from the first mobile device, a second RFID scan of an RFID tag affixed to a second mobile object; creating, in accordance with the first scan and the second RFID scan, a database relationship that identifies the second mobile object as being located at the first location inside the establishment; and storing the database relationship at a computer server system. The first input may include a first scan of a first tag affixed to the first location inside the establishment. The first scan may include an optical scan, and the first tag includes an encoded image.

### TECHNICAL FIELD

The present disclosure generally relates to object tracking and locating and more specifically to tracking and locating one or more objects within an establishment.

### BACKGROUND

Many types of equipment have become mobile these days. For example, an infusion pump may be carried from one hospital room to another; an X-ray scanner may be relocated from one hospital department to a different hospital department.

Difficulties abound, however, for locating and tracking mobile items: several items may take up an entire storage space, making it inconvenient to physically ascertain the presence (or absence) of each individual item; an item shared by several users may move among several different places, rendering it onerous to track its varying locations.

The above identified technical problems are reduced or eliminated by the apparatuses, systems, and methods disclosed in the present disclosure.

### BRIEF DESCRIPTION OF THE DRAWINGS

FIG. 1 is a block diagram illustrating an example system for object tracking and locating, according to some implementations.

FIG. 2 is a block diagram illustrating an example method for object tracking and locating, according to some implementations.

FIG. 3 is a block diagram illustrating an example system for scanning RFID tags, according to some implementations.

FIG. 4 is a block diagram illustrating an example method for scanning RFID tags, according to some implementations.

FIG. 5 is a block diagram illustrating an example system for object tracking and locating, according to some implementations.

FIG. 6 is a flowchart illustrating an example method for object tracking and locating, according to some implementations.

FIG. 7 is a block diagram illustrating an example system for object tracking and locating, according to some implementations.

FIG. 8 is a block diagram illustrating an example method for object tracking and locating, according to some implementations.

FIG. 9 is a block diagram illustrating an example method for object tracking and locating, according to some implementations.

FIG. 10 is a flowchart illustrating an example method for object tracking and locating, according to some implementations.

FIG. 11 is a block diagram illustrating an example scanning device, according to some implementations.

FIG. 12 is a block diagram illustrating an example computer system, according to some implementations.

Embodiments of the present disclosure and their advantages are best understood by referring to the detailed description that follows. It should be appreciated that like reference numerals are used to identify like elements illustrated in one or more of the figures; showings therein are for purposes of illustrating embodiments of the present disclosure and not for purposes of limiting the same.

### SUMMARY

Systems and methods for tracking and locating an object within an establishment are provided in the present disclosure.

In some implementations, a method for tracking and locating an object includes: receiving, from a first mobile device, a first input identifying a first location inside an establishment; receiving, from the first mobile device, a second RFID scan of an RFID tag affixed to a second mobile object; creating, in accordance with the first scan and the second RFID scan, a database relationship that identifies the second mobile object as being located at the first location inside the establishment; and storing the database relationship at a computer server system.

In some implementations, the first input includes a first scan of a first tag affixed to the first location inside the establishment.

In some implementations, the first scan includes an optical scan and the first tag includes an encoded image.

The encoded image includes, in some implementations, a bar code or a QR code.

In some implementations, the first scan includes a first RFID scan, and the first tag includes a first RFID tag.

The method, in some implementations, further comprises: causing to be displayed on the first mobile device a varying RFID signal strength between the RFID tag and the first mobile device.

In some implementations, the mobile device includes a scanning component and a detachable mobile computing device connected to the scanning component.

The detachable mobile computing device is, in some implementations, wirelessly connected to the scanning component.

The detachable mobile computing device is, in some implementations, connected to the scanning component through a BLUETOOTH connection.

The mobile computing device is, in some implementations, wirelessly coupled to a label printer.

The mobile computing device is, in some implementations, coupled to the label printer through a BLUETOOTH connection.

The method, in some implementation, further comprises: receiving, from the first mobile device, a third scan of a third tag affixed to a third location inside the establishment; receiving, from the first mobile device, a fourth scan of the second RFID tag affixed to the second mobile object; and updating the database relationship to identify the second mobile object as being located at the third location inside the establishment.

The computer server system is, in some implementations, located inside the establishment.

The computer server system is, in some implementations, a computing cloud server located outside the establishment.

An example method comprises: obtaining a first optical scan of a barcode affixed to a mobile object; determining first object identification data based on the first optical scan; causing, using a mobile application installed on a mobile computing device, a printer to print a second barcode onto a pre-programmed RFID tag; obtaining, using a reader device, an RFID tag scan of the pre-programmed RFID tag; receiving, into the mobile application, a tag identifier of the pre-programmed RFID tag; creating a database relationship associating the tag identifier with the first object identification data; and storing the database relationship in a database.

The database relationship includes, in some implementations, an association between the tag identifier and an encoded version of the first object identification data.

The first tag is, in some implementations, affixed to an entrance to a first location within a health care facility

The RFID tag is, in some implementations, an Ultra High Frequency (UHF) RFID tag.

A method comprising: receiving, from a first mobile device, an input designating a location; receiving, from the first mobile device, a second RFID scan of a plurality of RFID tags affixed to a plurality of mobile objects that are within a predefined distance from the first mobile device; creating, in accordance with the first input and the second RFID scan, a database relationship that identifies the second mobile object as being located at the first location inside the establishment; and storing the database relationship at a computer server system.

The predefined distance is, in some implementations, within 30 feet.

The plurality of RFID tags are, in some implementations, affixed to top surfaces of the plurality of mobile objects.

An example method comprises: receiving, a first scan of an RFID tag, from a first RFID antenna affixed to a first location within an establishment. The RFID tag is affixed to a first mobile object. The method further comprises: creating a first database relationship that identifies the first mobile object as being proximate to the first location within the establishment; storing the first database relationship at a computer server system; and receiving, a second scan of the RFID tag, from a second RFID antenna affixed to a second location within the establishment; and creating a second database relationship that identifies the first mobile object as being proximate to the second location within the establishment. The second location is different and has a predefined distance from the first location.

The first location is, in some implementations, a choke point.

The method, in some implementations, further comprises: storing the second database relationship in association with the first database relationship at the computer server system.

The first RFID reader, in some implementations, includes a plurality of antennas pointing in different directions associated with the first location.

The first RFID antenna and the second RFID antenna are, in some implementations, antennas associated with a same RFID reader, but pointing at two different directions.

The method, in some implementations, further comprises: determining that the first scan includes scanning the RFID tag by a first antenna in a plurality of antennas; and based on the determining, identifying, in the first database relationship, that first mobile object is moving away or towards the first location.

The method, in some implementations, further comprises: determining that the first scan includes scanning the RFID tag by a first antenna in a plurality of antennas; and based on the determining, identifying, in the first database relationship, that first mobile object is moving away or towards the first location.

Determining that the second scan includes scanning the RFID tag by a second antenna in the plurality of antennas; and the method, in some implementations, further comprises: determining a first direction associated with the first antenna; determining a second direction associated with the second antenna; and identifying, in the first database relationship, that first mobile object is moving from the first location towards to second direction.

The first RFID reader is, in some implementations, configured to continuously scan for RFID tags located within a predefined range from the first RFID reader.

The first RFID reader is, in some implementations, affixed to an entrance to or exit from the first location.

The first RFID reader is, in some implementations, a stationary reader.

An example method comprises: receiving, a first scan of a first RFID tag affixed to a mobile scanning device, from a first RFID reader affixed to a first location within an establishment; creating a first database relationship that identifies the first RFID mobile scanning device as being proximate to the first location within the establishment; sending the first location information to the mobile scanning device for a confirmation; receiving, from the mobile scanning device, a second scan of a second tag affixed to a second mobile object associated with the first location information; and storing a database relationship that associates the first RFID tag with the first location information at a computer server system.

The first location information, in some implementations, includes information from a location barcode tag.

The method, in some implementations, further comprises: receiving, a third scan of the first RFID tag affixed to the mobile scanning device, from a third RFID reader affixed to a third location within the establishment; creating a third database relationship that identifies the mobile scanning device as being proximate to the third location within the establishment; and creating a second database relationship that identifies the mobile scanning device as having been located at the first location and the third location.

The computer server system is, in some implementations, located inside the establishment.

The first RFID reader includes, in some implementations, two antennas pointing to two different directions associated with the first location.

The method, in some implementations, further comprises: determining that the first scan includes scanning the RFID tag by a first antenna in the two antennas; and based on the determining, identifying, in the first database relationship, that first mobile object is moving away or towards to first location.

A method, in some implementations, comprises: obtaining a first optical scan of a barcode affixed to a mobile object; retrieving, from a first database, first object identification data based on the first optical scan; causing, using a mobile application installed on a mobile computing device, a printer to print a second barcode onto a pre-programmed RFID tag; obtaining, using a reader device, an RFID tag scan of the pre-programmed RFID tag; receiving, into the mobile application, a tag identifier of the pre-programmed RFID tag; and creating a database relationship associating the tag identifier with the first object identification data; and storing the database relationship in a second database different form the first database.

A computing system method for tracking and locating one or more objects within an establishment as described in any of the implementations above.

A non-transitory computer readable medium comprising computer executable instructions stored thereon, which, when executed by one or more computers, cause a machine to track and locate one or more objects within an establishment as described in any of the implementations above.

### DETAILED DESCRIPTION

The implementations described herein provide various technical solutions to tracking and locating objects within an establishment and in particular to the above-identified problems by using either a walkabout system, or a chokepoint system, or a combination of both.

As an example, in a walkabout system, RFID tags may be affixed to different objects. A user may use a hand-held scanner to first scan a bar code which identifies a location within an establishment (e.g., the emergency room #1 at St. Jude hospital) and then scan a RFID system affixed to an infusion pump. Based on these two scans, a computer server may determine that the infusion pump is currently located at the location (e.g., the emergency room #1).

As another example, in a chokepoint system, RFID reader may be affixed to chokepoints. A chokepoint may be defined as a point of distinction between two areas or a convergence point between two areas, for example, a room entrance and a hallway intersection, respectively.

As a user moves an object from one location to another, the RFID tag affixed to the object is read by readers located between these two locations. A compute server may, based on each RFID read, determine a route along which that the object is moved, as well as locations where the object is left located.

The present disclosure describes various implementations of systems and methods for tracking and locating objects. The technologies described in the present disclosure can provide the following technical advantages. First, the presence of several objects may be detected by using a reader to conduct an RFID sweep of nearby objects, eliminating the need for a user to detect the individual item of each object. For example a user may use a RFID scanner to simultaneously read several RFID tags affixed to chairs stored in a storage room, rather than scanning the tag of each chair individually.

Second, the movements of an object may be tracked by different readers (or different antennas of a same reader) stationed at different locations, reducing the need for a user to affirmatively track the movements of the object. For example, as a mobile infusion pump is taken from one hospital department to another hospital department, readers located at hallway intersections can record the locations of the infusion pump and the recorded locations can be used to generate a location trail of the infusion pump.

Additional details of implementations are now described in relation to the Figures.

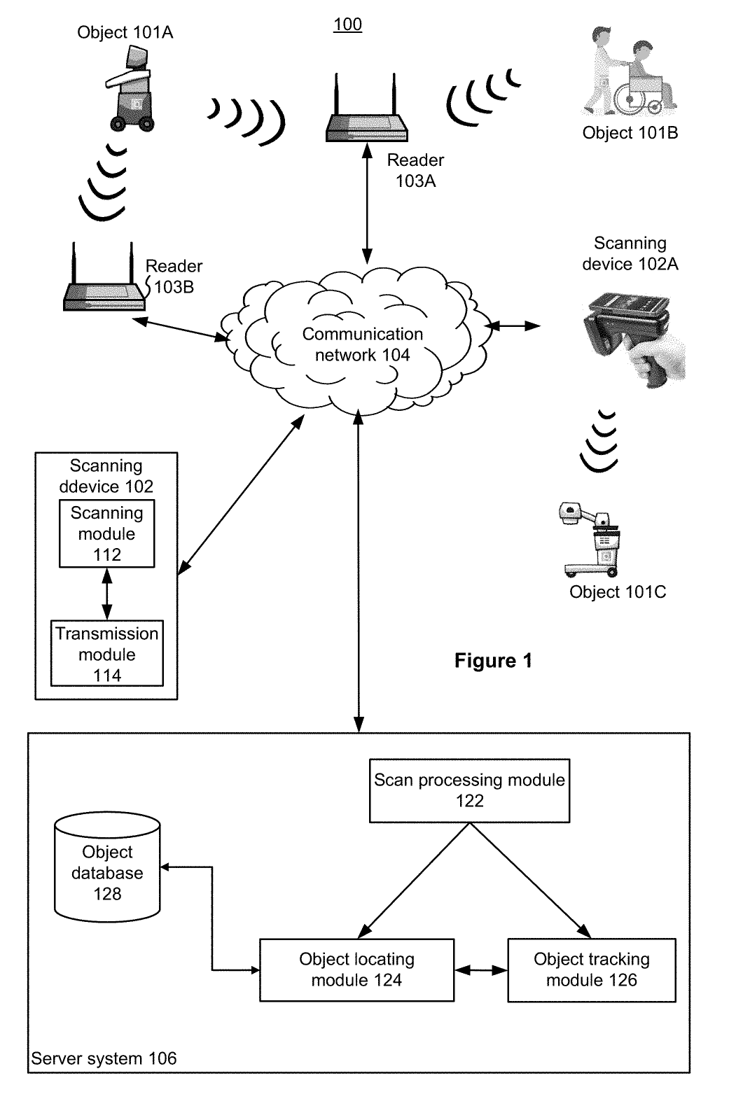

FIG. 1 is a block diagram illustrating an example system 100 for object tracking and locating, according to some implementations.

As shown in FIG. 1, the system 100 may include one or more mobile objects 101 (e.g., objects 101A, 101B, and 101C), a scanning device 102 (e.g., scanner 102A), one or more readers 103 (e.g., readers 103A and 103B), a communication network 104, and a computer server system 106.

A mobile object 101 may be a piece of office furniture (e.g., a file cabin or a stapler), a medical equipment (e.g., an infusion pump, an X-ray scanner, or a wheelchair), or a human person (e.g., a physician, a specialist, an ER technician, or a patient). A mobile object may move on its own (e.g., an autonomous moving cart) or be moved (e.g., carried or pushed) from one location from another.

A scanning device 102 (which may also be referred to as a scanner in the present disclosure) may scan text or images, such as barcodes, QR codes, photos, as well as Radio-Frequency IDentification (RFID) tags, such as Ultra High Frequency (UHF) RFID tags. A scanner may be a hand-held device, which may include a scanning module 112 and a transmission module 114. The scanning module 102 may optically read a barcode or a QR code or electronically read a RFID tag and decode information encoded in the barcode, in the QR code, or in the RFID tag.

The transmission module 114 may enable the scanner 102 to communicate with the server system 106, e.g., via the communication network 104 or via a short-range communication connection, such as a BLUETOOTH connection, a BLUETOOTH LOW ENERGY connection, or a Wi-Fi connection. For example, after decoding location information from an RFID read, the transmission module 114 may transmit the location information to the server system 106, where the location information is associated with an object.

In some implementations, the communication network 104 interconnects one or more scanners 102 with one or more readers 103, with the server system 106, and with each other. In some implementations, the communication network 104 optionally includes the Internet, one or more local area networks (LANs), one or more wide area networks (WANs), other types of networks, or a combination of such networks.

The server system (also referred to as a server in the present disclosure) 106 may communicated with a scanner 102 and a reader 103, via the communication network 104. For example, a scanner 102 may decode (1) an equipment identifier (e.g., the serial number of an X-ray machine) based on reading a barcode attached to the equipment and (2) a location identifier (e.g., a room number) based on reading an RFID tag affixed to a room entrance, and transmits the equipment identifier and the location identifier to the server 106. For another example, a scanner 102 may decode (1) a location identifier (e.g., a room number) based on reading a tag (e.g., a barcode or a QR code) affixed to a room entrance, (2) an equipment identifier (e.g., the serial number of an X-ray machine) based on reading an RFID tag attached to the equipment attached to the equipment, and transmits the equipment identifier and the location identifier to the server 106.

Upon receiving the equipment identifier and the location identifier, the server 106 may create a database entry to reflect the determination that the equipment is (or was) located at the location (e.g., the X-ray machine is currently located at the hospital operation room as identified by the room number).

The server 106 may include a scan processing module 122, an object locating module 124, an object tracking module 126, and an object database 128. The scan processing module 122 may decode information obtain from a scan, such as a barcode scan, a QR code scan or an RFID scan and provides the decoded information to the object locating modules 124 and to the object tracking module 126.

The object locating module 124 may, based on data received from the scanning processing module 122, determine the current (or past) location of an object. The object tracking module 126 may, based on one or more locations of an object, as determined by the object locating module, maintain a history of locations for the object. Location information, e.g., the current location or one or more past locations, of an object may be stored in the object database 128, in association with a unique identifier of the object.

In some implementations, a commissioning process is applied before implementing the walkabout or chokepoint technology.

For example, during a commissioning process, a user may scan an equipment's existing tag, which can be inside the device, or on an outer surface of the device, as in the case of a barcode tag, to retrieve information about the equipment from an existing third party database. For example, before implementing the walkabout or chokepoint technology at a hospital, mobile hospital equipment's make, model, and serial number may be retrieved from an existing file or database based on the scanning of an existing tag attached to the equipment. Equipment data retrieved from the existing file or database may be duplicated (and later updated) in the object database 128. The commissioning process provides an efficient initialization process for retrieving equipment data and a robust data management structure. Because, for example, access to a hospital database may become restricted, and the locating and tracking features may be weakened if access to equipment data becomes lacking. In another embodiment, the commissioning process includes assigning information to, or programming, OEM tags (tags that are incorporated into/onto the item during manufacture).

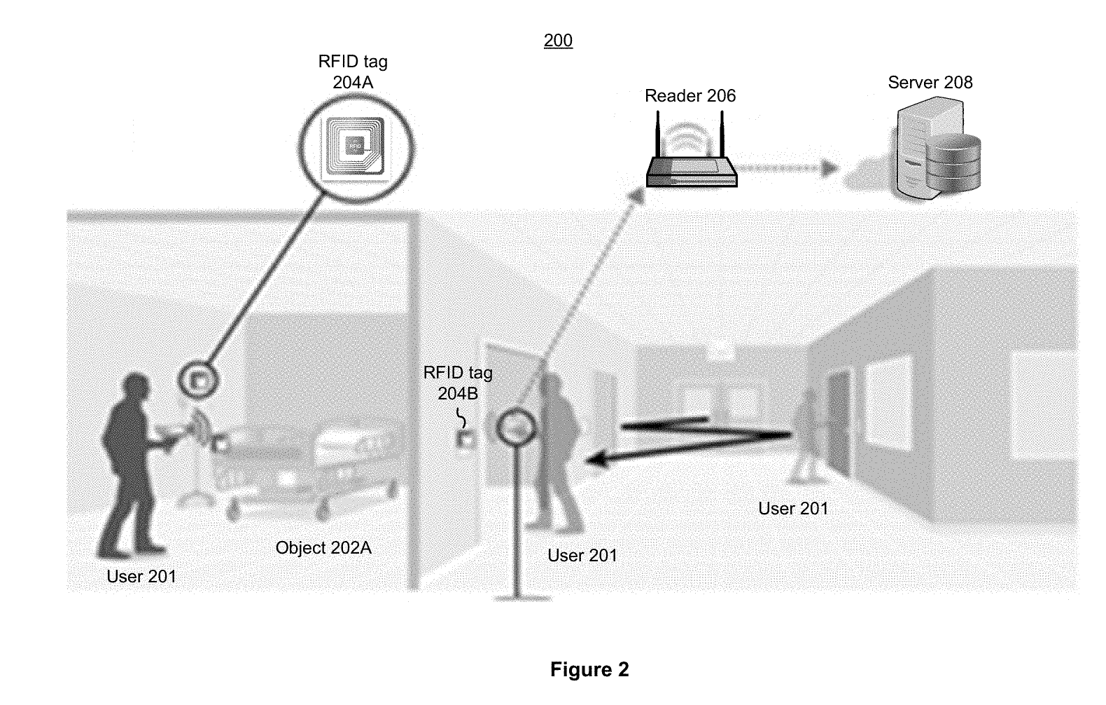

FIG. 2 is a diagram illustrating example methods 200 for object tracking and locating, according to some implementations.

As shown in FIG. 2, an object 202A may be a mobile hospital bed. To provide the current location of a hospital bed, a user 201 may, using a scanner, first scan a barcoded RFID tag (e.g., a barcode or a QR code printed on an outer surface of an RFID chip) attached to the hospital bed. In some implementations, an RFID tags are affixed to the outer surface of an object, e.g., to provide a better scan performances especially in a sweep scan (as explained in more detail below). In some other implementations, an RFID tags are affixed to an inner surface of an object, e.g., to provide added security, or because they are OEM tags.

An RFID tag may include a printable surface, such that data can be printed on the tag or a label may be affixed to the surface to facilitate a user inspection. In the implementations, where a label is affixed to a tag surface, the tag may need to meet a predefined set of lab protocols or medical equipment standards.

An RFID tag may be made of a thin and durable material, such as a laminate. An RFID may also include an adhesive surface such that the RFID may be reusable, capable of being removed from an objection after being affixed to an object, causing being damaged.

RFID tags may be made of different materials in order to be secure to different types of surfaces. For example, a hard surface tag may be used on a metal surface, while a RFID Wet Inlay may be used on a non-metal surface. Laminate or hard tags can be used exclusively or in combination in the system.

The scanner may decode data encoded in the barcode and in the RFID tag and transmit the decoded data to a reader 206, which in turn transmits the decoded data to the server 208.

Alternatively, the user 201 may, using a scanner, first scan an RFID tag 204B affixed near the entrance of a location. After entering the location (as identified by the RFID tag 208B), the user may scan either a barcode or an RFID tag 204A affixed to the object 202A (e.g., a hospital bed) located inside the location. The scanner may transmit the data obtained from these two scans to the server 208, via the reader 206 or using a cellular connection. Based on the data received from the scanner, the server 208 may determine the current location of the object 202A. Subsequent scans can automatically updated an item's location on a database without a user specifically requesting a location updated. For example, after a first scan and a second scan (e.g., a barcode scan and an RFID scan, respectively) are completed, an equipment location is determined and stored on a server; a third scan and a fourth scan (e.g., another barcode scan and another RFID scan, respectively) may update the equipment's location on the server (e.g., updating an equipment-location table on the server), if the server determines (A) that the first barcode scan and the second barcode scan reflect two different locations and (B) that both the first RFID scan and the second RFID scan represent the same equipment. For a second example, after a first scan and a second scan (e.g., a first RFID scan and a second RFID scan, respectively) are completed, an equipment location is determined and stored on a server; a third scan and a fourth scan (e.g., a third RFID scan and a fourth RFID scan, respectively) may update the equipment's location on the server (e.g., updating an equipment-location table on the server), if the server determines (A) that the first RFID scan and the third RFID scan reflect two different locations and (B) that both the second RFID scan and the fourth RFID scan represent the same equipment. This features reduces the need for a user to affirmatively request a location update, making it convenient to track and update a mobile object's location.

As shown in FIG. 2, the user 201 may walk around different locations within an establishment and scan RFID tags or barcodes affixed to objects at each location, technologies related to these implementations may thus also be referred to as walkabout technologies.

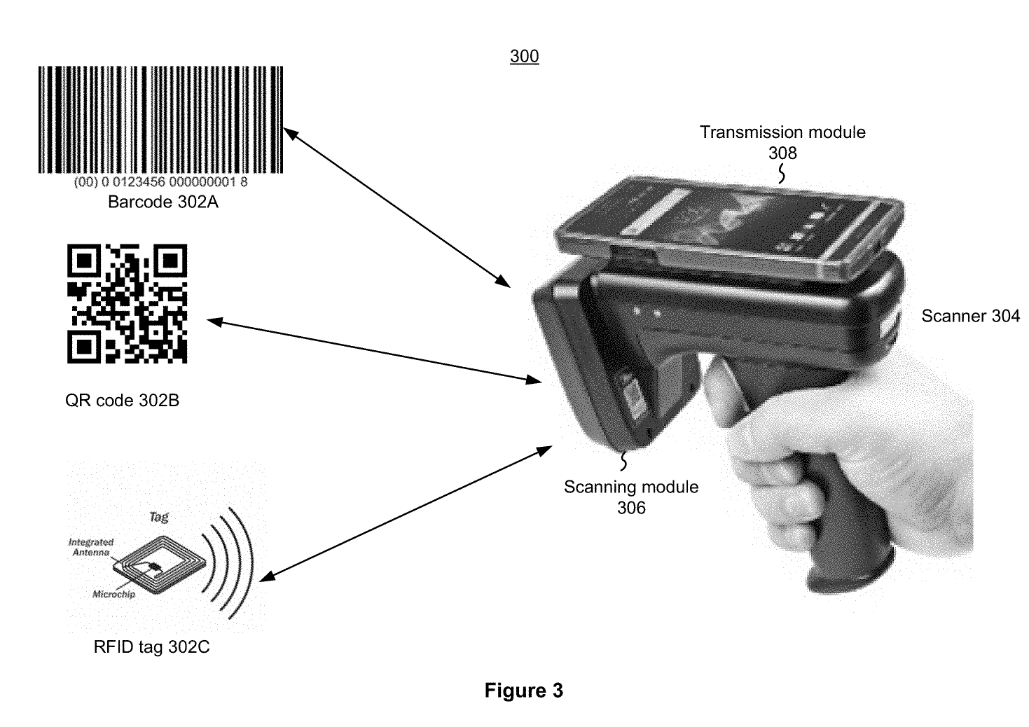

FIG. 3 is a diagram illustrating an example system 300 for scanning RFID tags, according to some implementations.

As shown in FIG. 3, the system 300 may include one or more codes or tags 302A-302C and a mobile scanner 304. The code 302A is a barcode; the code 302B is a QR code, the tag 302C is an RFID tag. A code or a tag may encode an equipment identifier or a location identifier.

As shown in FIG. 3, the mobile scanner 304 may be a hand-held scanner. The mobile scanner 304 may include a scanning module 306 and a transmission module 308. The transmission module 308 maybe a smartphone, which is detachable from the scanning module 306. Using a smartphone as a transmission module is technically advantageous, because it reduces the technical requirements on the scanning module. The scanning module may be equipped with basic scanning functionalities, the processing capacity needed for data decoding and transmission may be provided by the smartphone, which is often equipped with more computing power and multiple network connection capacities, compared to the scanning module 306. Shifting technical requirements to a smartphone may also help reduce the manufacturing and maintenance costs of a scanner.

It is noted that technologies described in the present disclosure may also be applied to the locating and tracking of the mobile scanner 304. For example, in a chokepoint system, an RFID tag may be affixed to a scanner. The scanner can then be located and tracked using the chokepoint technologies.

In some implementations, RFID signal strength may be displayed on the scanner 304. The signal strength may vary based on the distance between an RFID-tagged object and the scanner. The varying signal strength may serve as a direction guide for a user to physically locate RFID-tagged objects. For example, if the RFID signal for a medical pump increases as a user walks towards the entrance to a storage room, then it may be determined that the medical pump may be currently located near or inside the storage room.

Additionally, when the scanner includes a smartphone, the location of the smartphone (as determined by the smartphone's GPS component) may be used to facilitate the locating and tracking of mobile equipment. For example, the GPS location of the smartphone can be used to confirm or refine an equipment's location as determined based on an RFID scan alone.

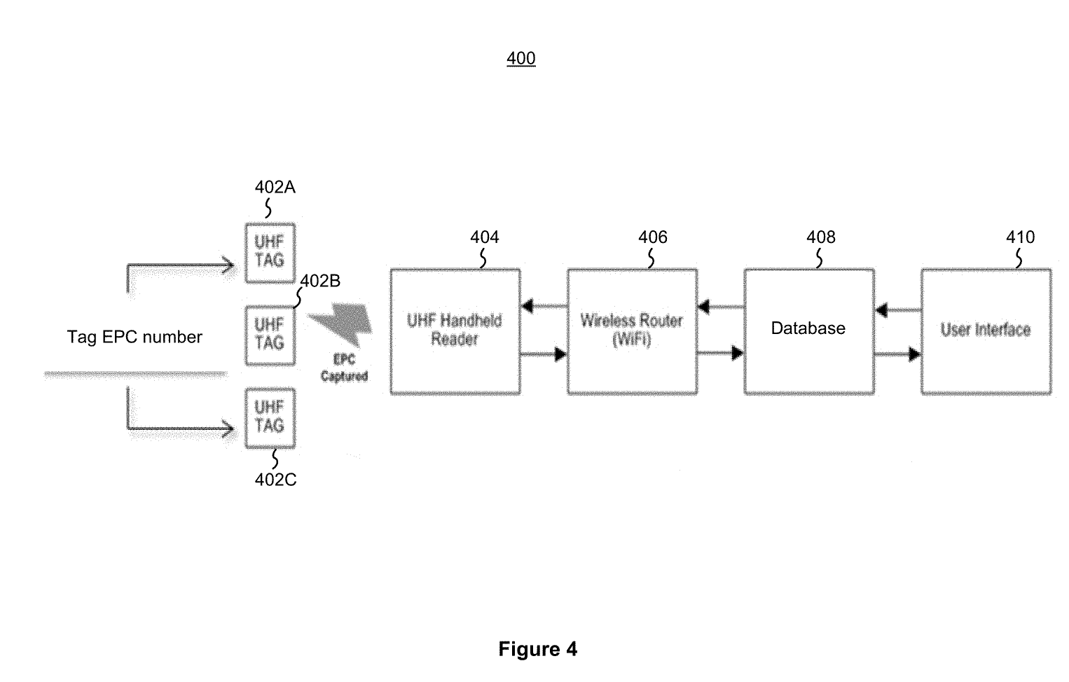

FIG. 4 is a block diagram illustrating an example method 400 for scanning RFID tags, according to some implementations.

In some implementations, a sweep scan feature may be provided. For example, a user may use a scanner to first scan a location barcode and then conduct a sweep scan of all detectable RFID tags (moving or stationary) within the scanner's detection range (for example 30 feet), rather than scanning each RFID tag individually. A server may then, in a wholesale fashion, assign the location (as determined from the location barcode) to all objects detected in the sweep scan, increasing the efficiency for locating and tracking a large number of mobile objects. In some implementations, a user may manually set a detection range (e.g., 30 feet) and conduct a sweep scan of all detectable RFID-tagged objects within the user-specified range.

As shown in FIG. 4, after a single sweep scan or several individual scans, a scanner (e.g., a UHF hand-held reader 404) captures Electronic Product Code (EPC) identifiers encoded in the RFID tags 402A-402C. An EPC identifier is a universal identifier that provides a unique identity for a physical object. The EPC structure is defined in the EPCglobal Tag Data Standard. Using EPC identifiers in connection with RFID technologies is technically advantageous, because the EPCglobal Tag Data Standard can provide a more compact data format for data storage and RFID tags typically have limited memory capacity, e.g., in order to keep the cost low.

The scanner may then transmit the captured EPC identifiers to a local wireless router 406, which further transmits the EPC identifiers to a database 408 provided on a server. Using a local Wi-Fi connection to transmit data from a scanner to a server database is technically advantageous, because it reduces the requirement that the scanner is enabled with a long-range connection, which may not be feasible at some remote sites, as well as providing higher data processing speed, as local data connections are often of higher and more reliable speed then remote data connections.

After processing the EPC identifiers and location identifiers captured from reading a barcode, a QR code, or another RFID tag, the server may provide feedback to a user interface 410 provided on the scanner.

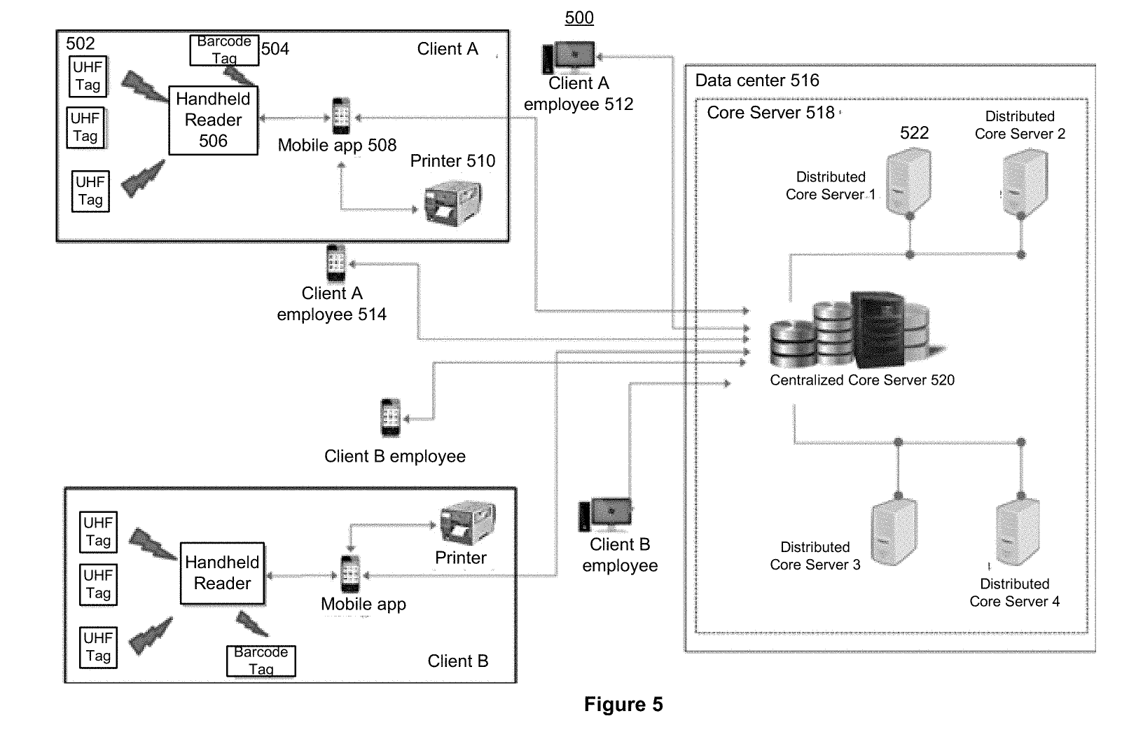

FIG. 5 is a block diagram illustrating an example system 500 for object tracking and locating, according to some implementations.

As shown in FIG. 5, a user may user a handheld reader 506 (also called a scanner) to scan either a barcode tag 504 or an RFID tag 502, or both. As explained with reference to FIG. 3, the scanner 506 may be equipped with a scanning module and a smartphone as the transmission module. The smartphone may be equipped with one or more software applications or apps 508 to process data obtained from a scan. The smartphone may be connected with a network printer 510 (e.g., a label printer) to facilitate data printout. The smartphone may be connected to the scanning module, to the network printer, or both, via a Wi-Fi connection or a BLUETOOTH connection.

Using a BLUETOOTH connection to connect different components of a scanner and to connect a scanner with a printer is technically advantageous for the following reasons. First, using a BLUETOOTH connection reduces reliance on a local Wi-Fi connection, which may not be available at certain facilities. Second, a single BLUETOOTH hub may be used to connect multiple types of devices simultaneously. Third, an external power source is not required to power a BLUETOOTH connection. Fourth, BLUETOOTH connections can provide better signal strength and reliability, in case of loss of power to Wi-Fi connections and variability of Wi-Fi signal.

Location or equipment identifiers obtained from the data scans may be transmitted to a remote data center 516 and stored on a core server 518. The core server 518 may include a centralized sever 520 and one or more distributed data servers 522. Different types of client devices, e.g., a desktop computer 512 and a smartphone 514, may be used to access the location data and equipment data stored at the data center 516.

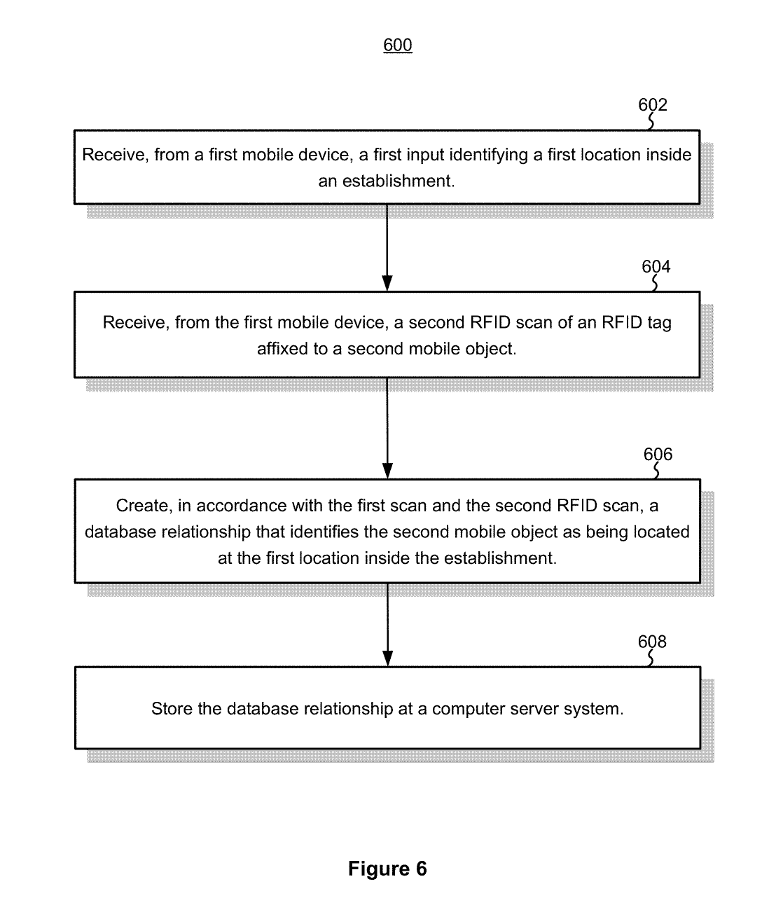

FIG. 6 is a flowchart illustrating an example method 600 for object tracking and locating 600, according to some implementations. The server 106, when properly programmed, can perform the method 600.

In some implementations, the method 600 for tracking and locating an object includes: receiving (602), from a first mobile device, a first input identifying a first location inside an establishment; receiving (604), from the first mobile device, a second RFID scan of an RFID tag affixed to a second mobile object; creating (606), in accordance with the first scan and the second RFID scan, a database relationship that identifies the second mobile object as being located at the first location inside the establishment; and storing (608) the database relationship at a computer server system.

In some implementations, the first input includes a first scan of a first tag affixed to the first location inside the establishment. In some implementations, the first scan includes an optical scan and the first tag includes an encoded image. The encoded image includes, in some implementations, a bar code or a QR code.

In some implementations, the first scan includes a first RFID scan, and the first tag includes a first RFID tag.

The method, in some implementations, further comprises: causing to be displayed on the first mobile device a varying RFID signal strength between the RFID tag and the first mobile device.

In some implementations, the mobile device includes a scanning component and a detachable mobile computing device connected to the scanning component. The detachable mobile computing device may be wirelessly connected to the scanning component. The detachable mobile computing device is, in some implementations, connected to the scanning component through a BLUETOOTH connection.

The scanning component is, in some implementations, wirelessly coupled to a label printer. The scanning component is, in some implementations, coupled to the label printer through a BLUETOOTH connection.

The method, in some implementation, further comprises: receiving, from the first mobile device, a third scan of a third tag affixed to a third location inside the establishment; receiving, from the first mobile device, a fourth scan of the second RFID tag affixed to the second mobile object; and updating the database relationship to identify the second mobile object as being located at the third location inside the establishment.

The computer server for tracking the location and movement of an object may be an onsite server or a remote server. For example, the computer server system may be located inside the establishment or a computing cloud server located outside the establishment.

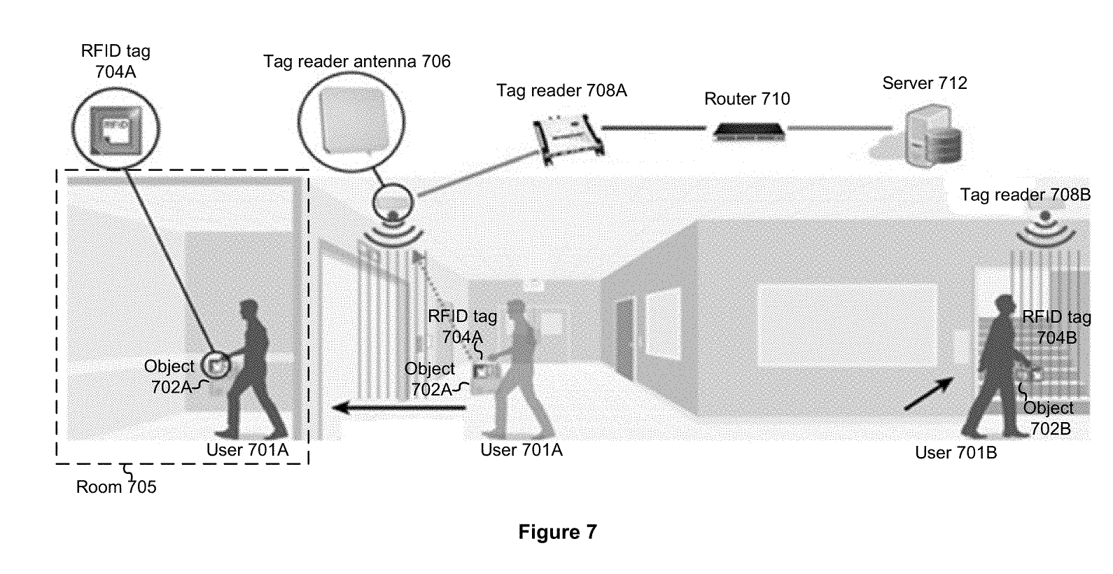

FIG. 7 is a diagram illustrating an example system for object tracking and locating, according to some implementations.

In addition to the walkabout technologies discussed with reference to, for example, FIG. 2, chokepoint technologies are also provided. For example, when a user 701A carries an object 702A from outside a hospital room 705 to inside the hospital room 705, a tag reader 708A located near the entrance (e.g., above the entrance) to the room may scan the RFID 704A attached to the object 702A.

The tag reader 708A may include a single antenna pointed at a particular direction or two or more antennas pointed at different directions. For example, the tag reader 708A may be equipped with a single antenna pointing towards inside the room 705 or the tag reader 708A may be equipped with two antennas, one pointing towards inside the room 705 and the other pointing towards outside the room 705. Based on which antenna performs the RFID scan on the RFID tag 704A, a reader may determine the movement direction of the object 702A.

For example, as shown in FIG. 7, as the user 701A moves the object 702A inside the hospital room 705, the RFID tag 704A becomes within range of the tag reader antenna 706. Because the antenna 706 points downwards, a scanning of the RFID tag 704A by the antenna 706 may be used to determine that the object 704 has entered or existed the hospital room 705.

Two or more readers may be used to track the movements of an object. For example, as shown in FIG. 7, as user 701B moves the object 702B outside the room 705 and towards another hospital location, the RFID tag 704 (affixed to the object 702) is read by the tag reader 708B, which is located near a stairway. Both the reader 708A and the reader 708B are connected via one or more routers 710 to the server 712.

Based on the first scans by the reader 708A and the second scan by the reader 708B, the server 712 may determine that the object 702B was taken first from outside the room 705 to inside the room 705 and then from inside the room 705 to outside the room 705, and to the stairway.

In some implementations, two or more different objects may be tracked at the same time, for example, the user 701A and 701B may carry different objects, which are tracked by readers 708A and 708B.

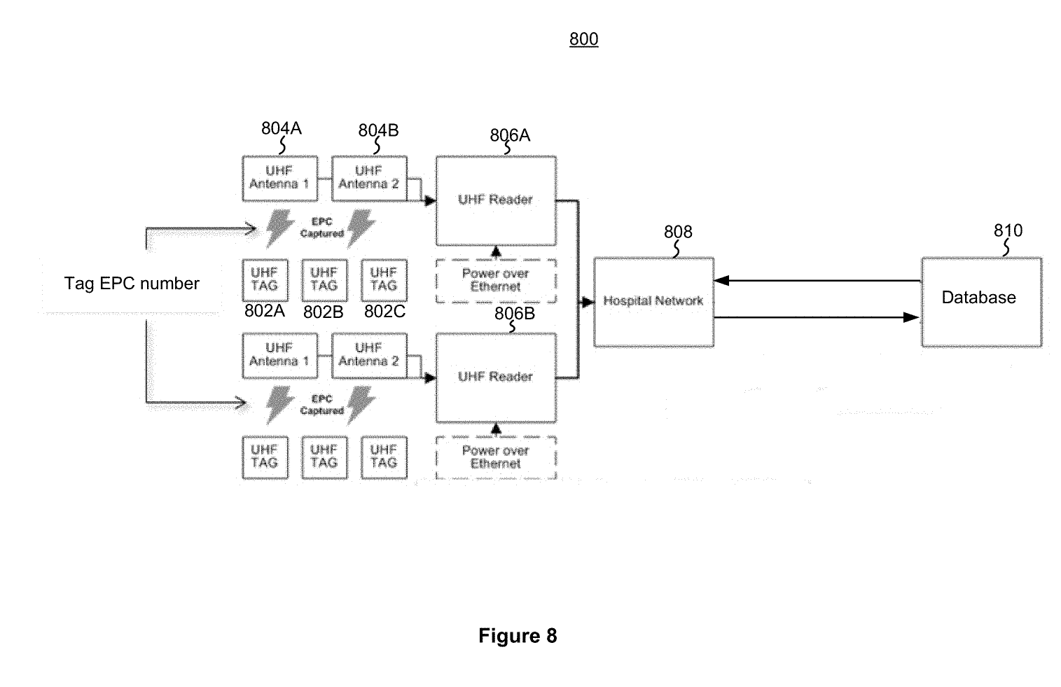

FIG. 8 is a block diagram illustrating an example method 800 for object tracking and locating, according to some implementations. The system 100 when properly programmed can execute the method 900.

The method 800 involves using a multi-antenna reader to track multiple objects. For example, tags 802A-802C (each of which is affixed to a different object) maybe read by the two antennas 804A and 804B of the reader 806A. As explained with reference to FIG. 7, the directions of the antennas 804A and 804B can be pre-setup and made known to a server. Based on which antennas (from a same reader or from different readers, e.g., the readers 806A and 806B) perform the scans of an RFID tag, the server can determine the movement direction of the object to which the RFID tag is attached. Similarly, the movements and locations of multiple objects may be tracked using a communication network provided at a hospital 808 and stored in a remote or local database 810.

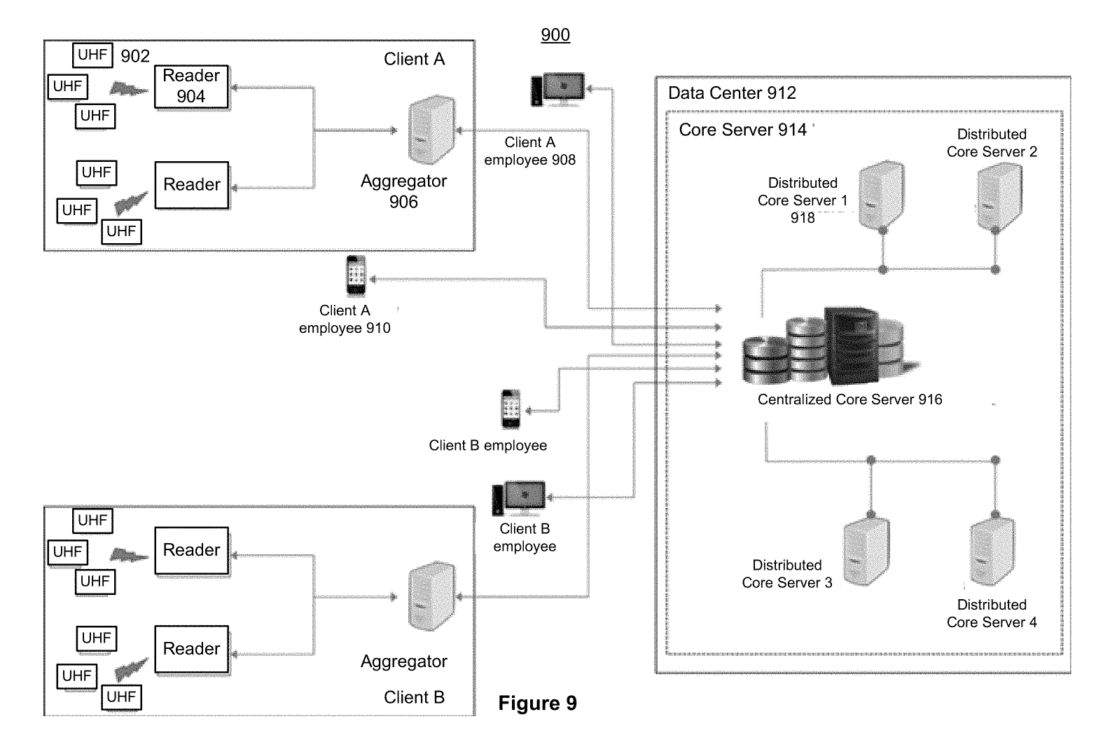

FIG. 9 is a block diagram illustrating an example method 900 for object tracking and locating, according to some implementations. The system 100 when properly programmed can execute the method 900.

As shown in FIG. 9, the locations and movements of multiple tags 902 and the corresponding objects may be simultaneously tracked using one or more stationary RFID readers 904. Scans performed by the stationary readers may be first aggregated at a local server 906, e.g., for better response time. Location and movement data from several local servers 906 may be further aggregated at a remote data center 912. The data center 912 may employee one or more core servers 914, each of which may include a centralized sever 916 and two or more distributed server 918.

By these ways, location and movement data of a large number of objects located at multiple facilities (e.g., hospitals) can be centrally managed at a remote data center, e.g., for data mining, as well as locally managed at a local server, e.g., for provider higher performance.

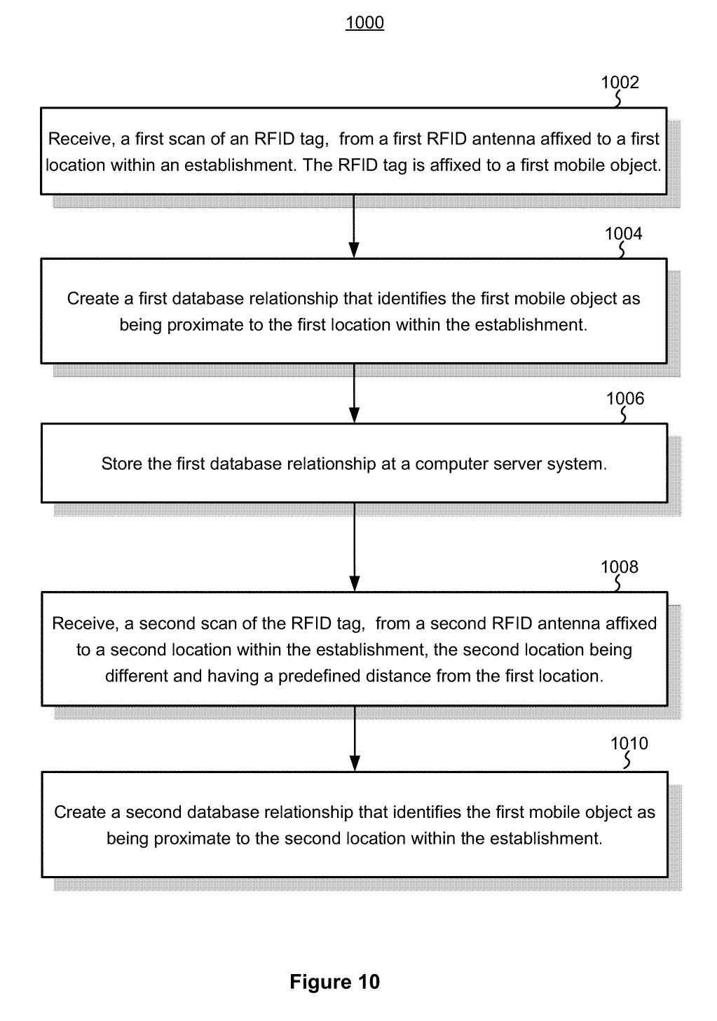

FIG. 10 is a flowchart illustrating an example method 1000 for object tracking and locating, according to some implementations. The server 106 when properly programmed can execute the method 900.

The method 1000 may include: receiving (1002), a first scan of an RFID tag, from a first RFID antenna affixed to a first location within an establishment. The RFID tag is affixed to a first mobile object. The method may further include: creating (1004) a first database relationship that identifies the first mobile object as being proximate to the first location within the establishment; storing (1006) the first database relationship at a computer server system; and receiving (1008), a second scan of the RFID tag, from a second RFID antenna affixed to a second location within the establishment; and creating (1010) a second database relationship that identifies the first mobile object as being proximate to the second location within the establishment. The second location is different and has a predefined distance from the first location. The first location is, in some implementations, a choke point.

The method, in some implementations, further comprises: storing the second database relationship in association with the first database relationship at the computer server system.

The first RFID reader, in some implementations, includes a plurality of antennas pointing in different directions associated with the first location.

The first RFID antenna and the second RFID antenna are, in some implementations, antennas associated with a same RFID reader, but pointing at two different directions.

The method, in some implementations, further comprises: determining that the first scan includes scanning the RFID tag by a first antenna in a plurality of antennas; and based on the determining, identifying, in the first database relationship, that first mobile object is moving away or towards the first location.

The method, in some implementations, further comprises: determining that the first scan includes scanning the RFID tag by a first antenna in a plurality of antennas; and based on the determining, identifying, in the first database relationship, that first mobile object is moving away or towards the first location.

Determining that the second scan includes scanning the RFID tag by a second antenna in the plurality of antennas; and the method, in some implementations, further comprises: determining a first direction associated with the first antenna; determining a second direction associated with the second antenna; and identifying, in the first database relationship, that first mobile object is moving from the first location towards to second direction.

The first RFID reader is, in some implementations, configured to continuously scan for RFID tags located within a predefined range from the first RFID reader.

The first RFID reader is, in some implementations, affixed to an entrance to or exit from the first location. The first RFID reader is, in some implementations, a stationary reader.

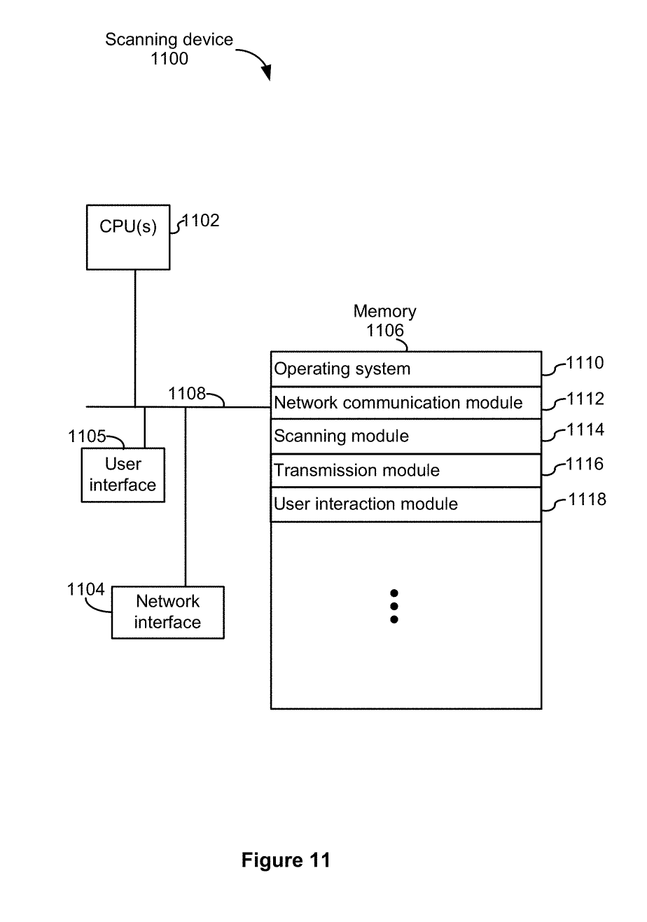

FIG. 11 is a block diagram illustrating an example scanning device 1100, according to some implementations. The scanning device 1100, in some implementations, includes one or more processing units CPU(s) 1102 (also referred to as processors), one or more network interfaces 1104, optionally a user interface 1105, a memory 1106, and one or more communication buses 1108 for interconnecting these components. The communication buses 1108 optionally include circuitry (sometimes called a chipset) that interconnects and controls communications between system components. The memory 1106 typically includes high-speed random access memory, such as DRAM, SRAM, DDR RAM or other random access solid state memory devices; and optionally includes non-volatile memory, such as one or more magnetic disk storage devices, optical disk storage devices, flash memory devices, or other non-volatile solid state storage devices. The memory 1106 optionally includes one or more storage devices remotely located from the CPU(s) 1102. The memory 1106, or alternatively the non-volatile memory device(s) within the memory 1106, comprises a non-transitory computer readable storage medium. In some implementations, the memory 1106 or alternatively the non-transitory computer readable storage medium stores the following programs, modules and data structures, or a subset thereof:
an operating system 1110 (e.g., an embedded Linux operating system), which includes procedures for handling various basic system services and for performing hardware dependent tasks;
a network communication module 1112 for connecting the scanning device 1100 with a server system via one or more network interfaces (wired or wireless);
a scanning module 1114 for scanning encoded data, e.g., a barcode or an RFID tag, affixed to an object;
a transmission module 1116 for transmitting data provided by the scanning module 1114 to a local or remote computing device; and
a user interaction module 1118 for enabling a user to interact with the scanning device 1100, e.g., via a Graphic User Interface (GUI).

In some implementations, the user interface 1105 includes an input device (e.g., a keyboard, a touch pad, and a touch screen) for a user to interact with the device 1100. A computing device may include a smartphone, a tablet computer, and a palm pilot device.

One or more of the above identified elements may be stored in one or more of the previously mentioned memory devices, and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 1106 optionally stores a subset of the modules and data structures identified above. Furthermore, the memory 1106 may store additional modules and data structures not described above.

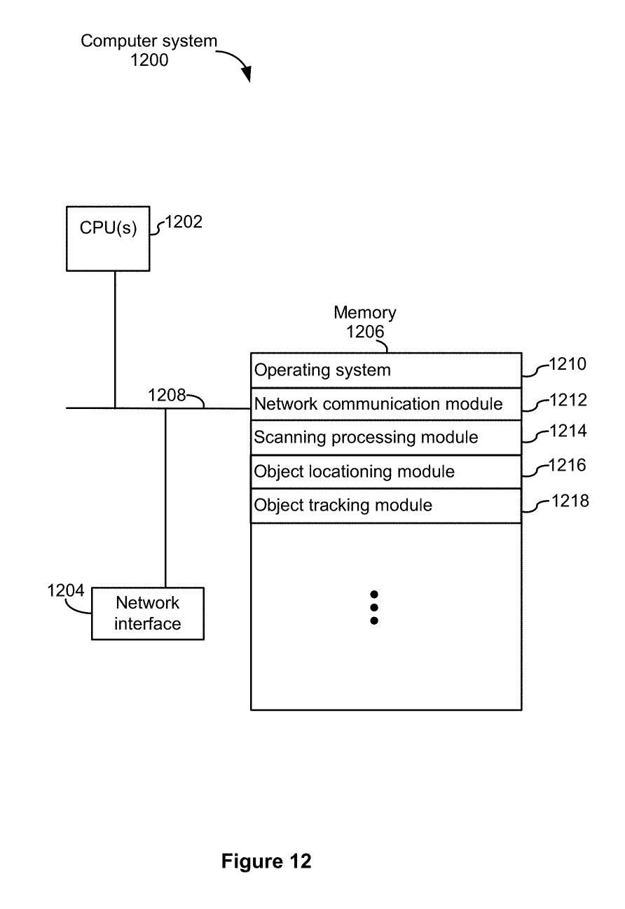

FIG. 12 is a block diagram illustrating an example computer system 1200, according to some implementations. The computer system 1200 in some implementations includes one or more processing units CPU(s) 1202 (also referred to as processors), one or more network interfaces 1204, a memory 1206, and one or more communication buses 1208 for interconnecting these components. The communication buses 1208 optionally include circuitry (sometimes called a chipset) that interconnects and controls communications between system components. The memory 1206 typically includes high-speed random access memory, such as DRAM, SRAM, DDR RAM or other random access solid state memory devices; and optionally includes non-volatile memory, such as one or more magnetic disk storage devices, optical disk storage devices, flash memory devices, or other non-volatile solid state storage devices. The memory 1206 optionally includes one or more storage devices remotely located from the CPU(s) 1202. The memory 1206, or alternatively the non-volatile memory device(s) within the memory 1206, comprises a non-transitory computer readable storage medium. In some implementations, the memory 1206 or alternatively the non-transitory computer readable storage medium stores the following programs, modules and data structures, or a subset thereof:
an operating system 1210 (e.g., an embedded Linux operating system), which includes procedures for handling various basic system services and for performing hardware dependent tasks;
a network communication module 1212 for connecting the computer system 1200 with a reader via one or more network interfaces (wired or wireless);
a scan processing module 1214 for decoding data obtained from a scan and for providing the decoded data to the object locating modules 1216 and to the object tracking module 1218;
an object locating module 1216 for determining the location, current or past, of an object based on data received from the scan processing module 1214; and
an object tracking module 1218 for determining and maintaining a history of locations for an object.

One or more of the above identified elements may be stored in one or more of the previously mentioned memory devices, and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 1206 optionally stores a subset of the modules and data structures identified above. Furthermore, the memory 1206 may store additional modules and data structures not described above.

Although FIGS. 11 and 12 show a “scanning device 1100” and a “computer system 1200,” respectively, FIGS. 11 and 12 are intended more as functional description of the various features which may be present in computer systems than as a structural schematic of the implementations described herein. In practice, and as recognized by those of ordinary skill in the art, items shown separately could be combined and some items could be separated.

In some implementations, a method for tracking and locating an object includes: receiving, from a first mobile device, a first input identifying a first location inside an establishment; receiving, from the first mobile device, a second RFID scan of an RFID tag affixed to a second mobile object; creating, in accordance with the first scan and the second RFID scan, a database relationship that identifies the second mobile object as being located at the first location inside the establishment; and storing the database relationship at a computer server system.

In some implementations, the first input includes a first scan of a first tag affixed to the first location inside the establishment.

In some implementations, the first scan includes an optical scan and the first tag includes an encoded image. The encoded image includes, in some implementations, a bar code or a QR code. In some implementations, the first scan includes a first RFID scan, and the first tag includes a first RFID tag.

The method, in some implementations, further comprises: causing to be displayed on the first mobile device a varying RFID signal strength between the RFID tag and the first mobile device.

In some implementations, the mobile device includes a scanning component and a detachable mobile computing device connected to the scanning component. The detachable mobile computing device is, in some implementations, wirelessly connected to the scanning component.

The detachable mobile computing device is, in some implementations, connected to the scanning component through a BLUETOOTH connection.

The scanning component is, in some implementations, wirelessly coupled to a label printer. The scanning component is, in some implementations, coupled to the label printer through a BLUETOOTH connection.

The method, in some implementation, further comprises: receiving, from the first mobile device, a third scan of a third tag affixed to a third location inside the establishment; receiving, from the first mobile device, a fourth scan of the second RFID tag affixed to the second mobile object; and updating the database relationship to identify the second mobile object as being located at the third location inside the establishment.

The computer server system is, in some implementations, located inside the establishment. The computer server system is, in some implementations, a computing cloud server located outside the establishment.

An example method comprises: obtaining a first optical scan of a barcode affixed to a mobile object; retrieving, from a first database, first object identification data based on the first optical scan; causing, using a mobile application installed on a mobile computing device, a printer to print a second barcode onto a pre-programmed RFID tag; obtaining, using a reader device, an RFID tag scan of the pre-programmed RFID tag; receiving, into the mobile application, a tag identifier of the pre-programmed RFID tag; creating a database relationship associating the tag identifier with the first object identification data; and storing the database relationship in a second database different form the first database.

The database relationship includes, in some implementations, an association between the tag identifier and an encoded version of the first object identification data. The first tag is, in some implementations, affixed to an entrance to the first location. The RFID tag is, in some implementations, an Ultra High Frequency (UHF) RFID tag.

The establishment is, in some implementations, a health care facility.

An example method comprises: receiving, a first scan of an RFID tag, from a first RFID antenna affixed to a first location within an establishment. The RFID tag is affixed to a first mobile object. The method further comprises: creating a first database relationship that identifies the first mobile object as being proximate to the first location within the establishment; storing the first database relationship at a computer server system; and receiving, a second scan of the RFID tag, from a second RFID antenna affixed to a second location within the establishment; and creating a second database relationship that identifies the first mobile object as being proximate to the second location within the establishment. The second location is different and has a predefined distance from the first location. The first location is, in some implementations, a choke point.

The method, in some implementations, further comprises: storing the second database relationship in association with the first database relationship at the computer server system.

The first RFID reader, in some implementations, includes a plurality of antennas pointing in different directions associated with the first location.

The first RFID antenna and the second RFID antenna are, in some implementations, antennas associated with a same RFID reader, but pointing at two different directions.

The method, in some implementations, further comprises: determining that the first scan includes scanning the RFID tag by a first antenna in a plurality of antennas; and based on the determining, identifying, in the first database relationship, that first mobile object is moving away or towards the first location.

The method, in some implementations, further comprises: determining that the first scan includes scanning the RFID tag by a first antenna in a plurality of antennas; and based on the determining, identifying, in the first database relationship, that first mobile object is moving away or towards the first location.

Determining that the second scan includes scanning the RFID tag by a second antenna in the plurality of antennas; and the method, in some implementations, further comprises: determining a first direction associated with the first antenna; determining a second direction associated with the second antenna; and identifying, in the first database relationship, that first mobile object is moving from the first location towards to second direction.

The first RFID reader is, in some implementations, configured to continuously scan for RFID tags located within a predefined range from the first RFID reader. The first RFID reader is, in some implementations, affixed to an entrance to or exit from the first location. The first RFID reader is, in some implementations, a stationary reader.

An example method comprises: receiving, a first scan of a first RFID tag affixed to a mobile scanning device, from a first RFID reader affixed to a first location within an establishment; creating a first database relationship that identifies the first RFID mobile scanning device as being proximate to the first location within the establishment; sending the first location information to the mobile scanning device for a confirmation; receiving, from the mobile scanning device, a second scan of a second tag affixed to a second mobile object associated with the first location information; and storing a database relationship that associates the first RFID tag with the first location information at a computer server system. The first location information, in some implementations, includes information from a location barcode tag.

The method, in some implementations, further comprises: receiving, a third scan of the first RFID tag affixed to the mobile scanning device, from a third RFID reader affixed to a third location within the establishment; creating a third database relationship that identifies the mobile scanning device as being proximate to the third location within the establishment; and creating a second database relationship that identifies the mobile scanning device as having been located at the first location and the third location.

The computer server system is, in some implementations, located inside the establishment. The first RFID reader includes, in some implementations, two antennas pointing to two different directions associated with the first location.

The method, in some implementations, further comprises: determining that the first scan includes scanning the RFID tag by a first antenna in the two antennas; and based on the determining, identifying, in the first database relationship, that first mobile object is moving away or towards to first location.

A method, in some implementations, comprises: obtaining a first optical scan of a barcode affixed to a mobile object; retrieving, from a first database, first object identification data based on the first optical scan; causing, using a mobile application installed on a mobile computing device, a printer to print a second barcode onto a pre-programmed RFID tag; obtaining, using a reader device, an RFID tag scan of the pre-programmed RFID tag; receiving, into the mobile application, a tag identifier of the pre-programmed RFID tag; and creating a database relationship associating the tag identifier with the first object identification data; and storing the database relationship in a second database different form the first database.

A method comprising: receiving, from a first mobile device, an input designating a location; receiving, from the first mobile device, a second RFID scan of a plurality of RFID tags affixed to a plurality of mobile objects that are within a predefined distance from the first mobile device; creating, in accordance with the first input and the second RFID scan, a database relationship that identifies the second mobile object as being located at the first location inside the establishment; and storing the database relationship at a computer server system.

The predefined distance is, in some implementations, 10 feet. The plurality of RFID tags are, in some implementations, affixed to top surfaces of the plurality of mobile objects.

A computing system method for tracking and locating one or more objects within an establishment as described in any of the implementations above.

A non-transitory computer readable medium comprising computer executable instructions stored thereon, which, when executed by one or more computers, cause a machine to track and locate one or more objects within an establishment as described in any of the implementations above.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first scan could be termed a second scan, and, similarly, a second scan could be termed a first scan, without changing the meaning of the description, so long as all occurrences of the “first scan” are renamed consistently and all occurrences of the “second scan” are renamed consistently. The first layer and the second layer are both layers, but they are not the same layer.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A method, comprising:
receiving, from a first mobile device, a first input identifying a first location inside an establishment;
receiving, from the first mobile device, a second RFID scan of an RFID tag affixed to a second mobile object;
creating, in accordance with the first scan and the second RFID scan, a database relationship that identifies the second mobile object as being located at the first location inside the establishment; and
storing the database relationship on a computer server system.

2. The method of claim 1, wherein the first input includes a first scan of a first tag affixed to the first location inside the establishment.

3. The method of claim 2, wherein the first scan includes an optical scan and the first tag includes an encoded image.

4. The method of claim 3, wherein the encoded image includes a bar code or a QR code.

5. The method of claim 2, wherein the first scan includes a first RFID scan and the first tag includes a first RFID tag.

6. The method of claim 1, further comprising: causing to be displayed on the first mobile device, a varying RFID signal strength between the RFID tag and the first mobile device.

7. The method of claim 1, wherein the first mobile device includes a scanning component and a detachable mobile computing device connected to the scanning component.

8. The method of claim 7, wherein the detachable mobile computing device is wirelessly connected to the scanning component.

9. The method of claim 8, wherein the detachable mobile computing device is connected to the scanning component through a BLUETOOTH connection.

10. The method of claim 7, wherein the mobile computing device is wirelessly coupled to a label printer.

11. The method of claim 10, wherein the mobile computing device is coupled to the label printer through a BLUETOOTH connection.

12. The method of claim 1, further comprising:
receiving, from the first mobile device, a third scan of a third tag affixed to a third location inside the establishment;
receiving, from the first mobile device, a fourth scan of the second RFID tag affixed to the second mobile object; and
updating the database relationship to identify the second mobile object as being located at the third location inside the establishment.

13. The method of claim 1, wherein the computer server system is located inside the establishment.

14. The method of claim 1, wherein the computer server system is a computing cloud server located outside the establishment.

15. A computing system, comprising:
one or more processors;
memory; and
one or more programs, wherein the one or more programs are stored in the memory and configured to be executed by the one or more processors, the one or more programs including instructions for:
receiving, from a first mobile device, a first input identifying a first location inside an establishment;
receiving, from the first mobile device, a second RFID scan of an RFID tag affixed to a second mobile object;
creating, in accordance with the first scan and the second RFID scan, a database relationship that identifies the second mobile object as being located at the first location inside the establishment; and
storing the database relationship at a computer server system.

16. The computing system of claim 15, wherein the first input includes a first scan of a first tag affixed to the first location inside the establishment.

17. The computing system of claim 16, wherein the first scan includes an optical scan and the first tag includes an encoded image.

18. The computing system of claim 17, wherein the encoded image includes a bar code or a QR code.

19. A non-transitory computer readable storage medium storing one or more programs, the one or more programs comprising instructions, which when executed by a computing system with one or more processors, cause the computing system to execute a method of:
receiving, from a first mobile device, a first input identifying a first location inside an establishment;
receiving, from the first mobile device, a second RFID scan of an RFID tag affixed to a second mobile object;
creating, in accordance with the first scan and the second RFID scan, a database relationship that identifies the second mobile object as being located at the first location inside the establishment; and
storing the database relationship at a computer server system.

20. The non-transitory computer readable storage medium of claim 19, wherein the first input includes an optical scan and the first tag includes a bar code or a QR code.
